(19) United States
(12) Patent Application Publication
(10) Pub. No.: US 2007/0150310 A1
(43) Pub. Date: Jun. 28, 2007

(54) A METHOD OF MOTIVATING AN INDIVIDUAL TO IMPROVE LIFESTYLE FACTORS

(76) Inventors: Benjamin Wiegand, Yardley, PA (US); Kathryn Luedtke, Doylestown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/318,791

(22) Filed: Dec. 27, 2005

(51) Int. Cl. G06Q 50/00; C12Q 1/00

(52) U.S. Cl. 705/2; 435/4

(57) ABSTRACT

The invention relates to a method of assessing the pro-inflammatory immune health of a healthy individual comprising: (a) establishing a benchmark pro-inflammatory protein value set, wherein the pro-inflammatory protein value is determined by a calculation made using levels of pro-inflammatory protein measured in samples of non-blood body fluid collected from healthy individuals at one or more time points in a 24 hour period; (b) determining an optimal pro-inflammatory protein value range from the benchmark pro-inflammatory protein value set established in (a) by using descriptive statistic methodology; (c) establishing a pro-inflammatory protein value for said individual to be assessed, wherein said value is determined by the same calculation as set forth in (a); and (d) comparing the value established in step (c) to the optimal pro-inflammatory protein value range determined in step (b), wherein a pro-inflammatory protein value for said individual that is within the optimal pro-inflammatory protein value range is indicative of good pro-inflammatory immune health and wherein a pro-inflammatory protein value that is outside the optimal pro-inflammatory protein value range is indicative of inferior pro-inflammatory immune health.

### FIELD OF THE INVENTION

The invention relates to a method of assessing the pro-inflammatory immune health of a healthy individual comprising:
(a) establishing a benchmark pro-inflammatory protein value set, wherein the pro-inflammatory protein value is determined by a calculation made using levels of pro-inflammatory protein measured in samples of non-blood body fluid collected from healthy individuals at one or more time points in a 24 hour period;
(b) determining an optimal pro-inflammatory protein value range from the benchmark pro-inflammatory protein value set established in (a) by using descriptive statistic methodology;
(c) establishing a pro-inflammatory protein value for said individual to be assessed, wherein said value is determined by the same calculation as set forth in (a); and
(d) comparing the value established in step (c) to the optimal pro-inflammatory protein value range determined in step (b), wherein a pro-inflammatory protein value for said individual that is within the optimal pro-inflammatory protein value range is indicative of good pro-inflammatory immune health and wherein a pro-inflammatory protein value that is outside the optimal pro-inflammatory protein value range is indicative of inferior pro-inflammatory immune health.

### BACKGROUND OF THE INVENTION

Inflammation is the process by which the body reacts to injury, illness and disease. A counter-regulatory feedback loop exists between the immune and central nervous systems in which the body, in response to injury and/or disease, produces certain proteins such as c-reactive protein (“CRP”) and cytokines such as interleukin 6 (“IL-6”). These proteins serve to regulate inflammatory responses in the body and are termed pro-inflammatory proteins. Accordingly, pro-inflammatory proteins can serve as biomarkers of the level of inflammation or pro-inflammatory activity in the body.

Acute, i.e., a short duration, inflammation, e.g., response to a specific cellular injury, illness or disease that is marked by capillary dilatation, leukocytic infiltration, redness, heat, and/or pain, serves as a mechanism initiating the elimination of noxious agents, for example, from diseases and of damaged tissues caused by injury. Once the specific cellular injury, illness or disease is managed, the level of inflammation returns to its normal level. Accordingly, acute inflammation can have beneficial merits.

Chronic, low grade inflammation, on the other hand, is long term, inflammation characterized by an increase in the basal level of pro-inflammatory activity. Recently, researchers have discovered that chronic, low-grade inflammation can occur across multiple body systems in the absence of a specific cellular injury, illness or disease. See, for example, Silent Inflammation by Barry Sears, Nutraceuticals World, May 1, 2005, the disclosure of which is hereby incorporated by reference. Numerous lifestyle factors such as chronic stress, high body mass index, lack of sleep, poor quality of sleep, smoking, caffeine consumption, and alcohol consumption have been implicated in causing chronic, low-grade inflammation. Further discussion of low grade inflammation and its relationship to lifestyle factors can be found in the following articles, all of which are hereby incorporated by reference: Esposito et al. Nutr Metab. Cardiovasc Dis. 2004 October; 14(5): 228-32; Aygum et al. Mediators Inflamm. 2005 August; 2005(3); 180-3; Niklas et al. CMAJ 2005 April 26; 172(9): 1199-209. This chronic increase in the basal level of pro-inflammatory activity affects immune health and has been linked to numerous health conditions such as cancer, heart disease, diabetes, chronic skin disorders, asthma, allergy, arthritis, mental/behavioral illness, auto-immune disorders (lupus, MS etc.). The various causes and implications of pro-inflammatory activity are detailed in The Anti-Inflammation Zone: Reversing the Silent Epidemic That's Destroying Our Health, Chapters 2-3, Barry Sears, 2005 ,the disclosure which is hereby incorporated by reference. Accordingly, it would be beneficial to assess the pro-inflammatory immune health of an individual by measuring the level of pro-inflammatory activity of the individual.

International Publication No. WO 91/04479 relates to a diagnostic test for testing susceptibility of individuals to inflammatory diseases. The method comprises the steps of administering to a mammal a compound which is effective in stimulating the hypothalamic pituitary adrenal (HPA) axis and measuring the level of hormones secreted by the pituitary and adrenal gland of the mammal. This method requires blood collection and the stimulation of the HPA axis by the administration of a compound such as cytokines, cell growth factors, neuroendocrine hormones etc. There is no teaching or suggestion of a non-invasive method for measuring pro-inflammatory activity of a non-stimulated healthy individual. Non-stimulated methods are preferred because they are less intrusive to the individual being assessed and thus are more representative of their typical health status. Additionally, stimuli like pharmaceutical interventions put the individual at risk for adverse reactions and side effects.

U.S. Pat. Nos. 5,965,379 and 5,587,294 relate to methods for measuring cytokines in the blood as well as in saliva and nasal secretions. These patents fail to teach or suggest a method for assessing immune health that recognizes and utilizes a reliable secretory profile of pro-inflammatory protein.

A reliable secretory profile of pro-inflammatory protein would enable the establishment of a benchmark pro-inflammatory protein value set which could be used to assess the immune health of an individual without the need for establishing a baseline for each individual to be measured. The establishment of a baseline profile would be necessary in the absence of a benchmark pro-inflammatory value set since each person is different in terms of their basal pro-inflammatory protein levels across the day. Establishing the baseline profile is inconvenient since you need to measure the individuals profile at one point in time and then measure it again later in order to see what had changed. Additionally, this baseline method does not allow for an understanding of the status of the individuals level at the initial baseline and is thus limited in its utility. Accordingly, there exists a need for a non-invasive method of assessing the pro-inflammatory immune health of an individual without the need for establishing a baseline profile for each individual to be measured. The present invention answers this need.

### SUMMARY OF THE INVENTION

Many pro-inflammatory proteins have diurnal rhythms, i.e., the circulating level of protein in the body of mammals changes in a predictable pattern over a 24-hour period of time which is divided into two main periods, one a period of wakefulness and the other a period of sleepfulness. Thus, in order to understand the secretory profile of these proteins, we have discovered that it is necessary to collect sample(s) at one or more time points throughout the day relative to the time of morning waking throughout the wakeful period. The non-invasive nature of saliva allows for less stressful multiple collections throughout the day. However, as with any assessment protocol, simple and short is better since assessments interfere with the individual's schedule and are somewhat unpleasant to do. Thus, even with saliva collection, it is still necessary to minimize the number of samples collected It has been discovered that when samples are collected with respect to morning waking, it is possible to establish a reliable secretory profile with a small number of samples, thus minimizing the burden and stress placed on the individual.

It has also been discovered that a benchmark pro-inflammatory protein value set for optimal levels of pro-inflammatory protein can be established by measuring the level of pro-inflammatory protein in non-blood body fluid of healthy individuals at certain times throughout the day. Once established, the benchmark pro-inflammatory protein value set provides a tool for more accurately assessing the pro-inflammatory immune health of an individual. The level of pro-inflammatory protein in non-blood body fluid of an individual can be compared to the range of levels of pro-inflammatory protein in the benchmark pro-inflammatory protein value set, and thus the pro-inflammatory immune health of the individual can be determined, without requiring the establishment of a baseline profile of pro-inflammatory protein level for each individual to be assessed and without the need for stimulation.

Accordingly, the invention relates to a method of assessing the pro-inflammatory immune health of a healthy individual comprising:
(a) establishing a benchmark pro-inflammatory protein value set, wherein the pro-inflammatory protein value is determined by a calculation made using levels of pro-inflammatory protein measured in samples of non-blood body fluid collected from healthy individuals at one or more time points in a 24 hour period;
(b) determining an optimal pro-inflammatory protein value range from the benchmark pro-inflammatory protein value set established in (a) by using descriptive statistic methodology;
(c) establishing a pro-inflammatory protein value for said individual to be assessed, wherein said value is determined by the same calculation as set forth in (a); and
(d) comparing the value established in step (c) to the optimal pro-inflammatory protein value range determined in step (b), wherein a pro-inflammatory protein value for said individual that is within the optimal pro-inflammatory protein value range is indicative of good pro-inflammatory immune health and wherein a pro-inflammatory protein value that is outside the optimal pro-inflammatory protein value range is indicative of inferior pro-inflammatory immune health.

It has also been discovered that a more accurate and reliable assessment can be achieved by calculating the value of pro-inflammatory protein at least at two time points in a 24 hour period. Accordingly, in another embodiment, the invention relates to method of assessing the pro-inflammatory immune health of a healthy individual comprising:
(a) establishing a benchmark pro-inflammatory protein value set, wherein the pro-inflammatory protein value is determined by a calculation made using levels of pro-inflammatory protein measured in samples of non-blood body fluid collected from healthy individuals at two or more time points in a 24 hour period;
(b) determining an optimal pro-inflammatory protein value range from the benchmark pro-inflammatory protein value set established in (a) by using descriptive statistic methodology;
(c) establishing a pro-inflammatory protein value for said individual to be assessed, wherein said value is determined by the same calculation as set forth in (a); and
(d) comparing the value established in step (c) to the value determined in step (b), wherein a pro-inflammatory protein value for said individual that is within the optimal pro-inflammatory protein value range is indicative of good pro-inflammatory immune health and wherein a pro-inflammatory protein value outside the optimal pro-inflammatory protein value range is indicative of inferior pro-inflammatory immune health.

In yet another embodiment, the invention relates to a method for recommending a program to improve or maintain pro-inflammatory immune health of a healthy individual. The method comprises:
(a) establishing a benchmark pro-inflammatory protein value set wherein the pro-inflammatory protein value is determined by a calculation made using levels of pro-inflammatory protein measured in samples of non-blood body fluid collected from healthy individuals at one or more time points in a 24 hour period;
(b) determining an optimal pro-inflammatory protein value range from the benchmark pro-inflammatory protein value set established in (a) by using descriptive statistic methodology;
(c) establishing a pro-inflammatory protein value for said individual to whom a program will be recommended, wherein said value is determined by the same calculation set forth in (a); and
(d) comparing the value established in step (c) to the optimal pro-inflammatory protein value range determined in step (b), wherein a pro-inflammatory protein value for said individual that is within the optimal pro-inflammatory protein value range is indicative of good pro-inflammatory immune health and wherein a pro-inflammatory protein value that is outside the optimal pro-inflammatory protein value range is indicative of inferior pro-inflammatory immune health;
(e) wherein when the pro-inflammatory protein value for said individual is within the optimal pro-inflammatory protein value range, a program that comprises instructions for the individual to continue practicing his or her current lifestyle factors is recommended;
(f) wherein when the pro-inflammatory protein value for said individual is outside the optimal pro-inflammatory protein value range, a program that comprises instructions for the individual to change current lifestyle factors;
   In another embodiment, the invention relates to a method for assessing the efficacy of a program to improve or maintain the pro-inflammatory immune health of a healthy individual comprising: prior to the start of said program, assessing the baseline pro-inflammatory immune health of said individual by:
   (i) establishing a benchmark pro-inflammatory protein value set wherein the pro-inflammatory protein value is determined by a calculation made using levels of pro-inflammatory protein measured in samples of non-blood body fluid collected from healthy individuals at one or more time points in a 24 hour period;
   (ii) determining an optimal pro-inflammatory protein value range from the benchmark pro-inflammatory protein value set established in (i) by using descriptive statistic methodology;
   (iii) establishing a pro-inflammatory protein value for said individual for whom the efficacy of a program to improve pro-inflammatory immune health is being assessed, wherein said value is determined by the same calculation set forth in (i); and
   (iv) comparing the value established in step (iii) to the optimal pro-inflammatory protein value range determined in step (ii), wherein a pro-inflammatory protein value for said individual that is within the optimal pro-inflammatory protein value range is indicative of good pro-inflammatory immune health and wherein a pro-inflammatory protein value that is outside the optimal pro-inflammatory protein value range is indicative of inferior pro-inflammatory immune health.
(b) after the start of said program to improve or maintain pro-inflammatory immune health of a healthy individual, assessing the post-program pro-inflammatory immune health of said individual according to steps (i) to (iv) set forth in (a);
(c) comparing the post-program pro-inflammatory immune health assessed in step (b) to the baseline pro-inflammatory immune health assessed in step (a);
(d) wherein a change from inferior pro-inflammatory immune health to good pro-inflammatory immune health is an indication that the program is efficacious.

In another embodiment, the invention relates to a method of motivating an individual to improve lifestyle factors. The method comprises:
(a) assessing the pro-inflammatory immune health of a healthy individual comprising:
   (i) establishing a benchmark pro-inflammatory protein value set, wherein the pro-inflammatory value is determined by a calculation made using levels of pro-inflammatory protein measured in samples of non-blood body fluid collected from healthy individuals at two or more time points in a 24 hour period;
   (ii) determining an optimal pro-inflammatory protein value range from the benchmark pro-inflammatory protein value set established in (i) by using descriptive statistic methodology;
   (iii) establishing a pro-inflammatory protein value for said individual to be assessed, wherein said value is determined by the same calculation as set forth in (i); and
   (iv) comparing the value in step (iii) to the value in step (ii), wherein a pro-inflammatory protein value for said individual that is within the optimal pro-inflammatory protein value range is indicative of good pro-inflammatory immune health and wherein a pro-inflammatory protein value outside the optimal pro-inflammatory protein value range is indicative of inferior pro-inflammatory immune health;
(b) reporting the comparison of (iv) to said individual, wherein said individual is motivated by said comparison to improve, change, or maintain lifestyle factors.

In another embodiment, the invention relates to a kit comprising
a.) at least two saliva sample collection tubes;
b.) instructions on how to collect, store and ship saliva samples, wherein said instructions require the collection of saliva samples at least at one time point relative to morning waking.

### BRIEF DESCRIPTION OF THE DRAWINGS

FIG. 1: FIG. 1 is a representation of a typical salivary IL-6 profile illustrating important time points for saliva collection.

FIG. 2: FIG. 2 illustrates an example of the area under the curve defined by the level of pro-inflammatory protein, in this case interleukin-6, at time of waking (T0), 30 minutes after waking (T30), 10 hours after waking (T10H), and 16 hours after waking (T16H). The area under the curve is represented by the shaded area.

FIG. 3: FIG. 3 illustrates the average salivary IL-6 levels across four days for the individuals assessed in Example I.

FIG. 4: FIG. 4 illustrates the average salivary IL-6 levels taken at time of waking, at time of waking, 30 minutes after waking, 60 minutes after waking, 10 hours after waking, and 16 hours after waking for the individuals assessed in Example I.

FIG. 5: FIG. 5 illustrates the scatter plot of sleep time versus morning salivary IL-6 levels where morning salivary IL6 was calculated as the area under the curve defined by salivary IL6 levels at T0 (morning waking), T30 (thirty minutes post waking) and T60 (sixty minutes post waking) as described in Example II. FIG. 6: FIG. 6 illustrates a benchmark data set & 10th percentiles for slope of pro-inflammatory protein from T0 (morning waking) to T30 (thirty minutes post waking).

### DETAILED DESCRIPTION OF THE INVENTION

Definitions

As used herein, “immune health” means the ability of the immune system to function, respond and perform as it is intended.

As used herein, “individuals” include any of a class of warm-blooded higher vertebrates that nourish their young with milk secreted by mammary glands and have skin usually more or less covered with hair, and non-exclusively includes humans, dogs and cats.

As used herein, “healthy individual” means an individual who does not have any known chronic, immune-related medical conditions including autoimmune disorders, chronic illness, or other self-identified chronic condition including but not limited to rheumatoid arthritis, lupus, multiple sclerosis, vasculitis, HIV-1, hepatitis, hematologic disorders, type 1 diabetes, cancer, asthma or inflammatory bowel disease, and is not undergoing pharmaceutical treatment and not subjected to acute physical, emotional or psychological stimulus intended to change pro-inflammatory activity.

As used herein, “non-invasive” means collected from the individual without puncturing the skin or inserting anything into an orifice.

As used herein the term “non-blood body fluid” means any body fluid of an individual that may contain pro-inflammatory proteins and which can be obtained in a non-invasive manner. Examples of non-blood body fluids include saliva, nasal secretions and breath.

As used herein, “benchmark pro-inflammatory protein value set” means at least the minimum number of pro-inflammatory protein values needed to establish a normal distribution. As used herein, “normal distribution” means a distribution of the density of a set of values that is approximately symmetric and has an approximately bell-shaped density curve with a single peak, as tested by fulfilling the requirements of a normality test.

As used herein, the term “pro-inflammatory protein value” means the result of a calculation made using levels of pro-inflammatory protein measured in samples of non-blood body fluid collected from healthy individuals at one or more time points in a 24 hour period, wherein the calculation could be as simple as measuring the level of pro-inflammatory protein in non-blood body fluid of samples collected at one time point in a 24 hour period.

As used herein, “waking” means the time when an individual is first roused from nighttime sleep, but before rising from bed, showering, dressing, brushing teeth or completing other post-nighttime sleep tasks.

The term “about” in reference to time points, including waking, means the time recited plus or minus 30 minutes, including plus or minus 15 minutes, including plus or minus 10 minutes.

As discussed above, in one embodiment, the invention relates to a method of assessing the pro-inflammatory immune health of a healthy individual comprising:
(a) establishing a benchmark pro-inflammatory protein value set wherein said pro-inflammatory protein value is determined by a calculation made using levels of pro-inflammatory protein measured in samples of non-blood body fluid collected from healthy individuals at one or more time points in a 24 hour period;
(b) determining an optimal pro-inflammatory protein value range from the benchmark pro-inflammatory protein value set established in (a) by using descriptive statistic methodology;
(c) establishing a pro-inflammatory protein value for said individual to be assessed by measuring the level of the pro-inflammatory protein in non-blood body fluid of said individual, wherein the level of pro-inflammatory protein is measured at least at one same time point as measured in the benchmark pro-inflammatory protein value set; and
(d) comparing the value established in step (c) to the optimal pro-inflammatory protein value range determined in step (b), wherein a pro-inflammatory protein value for said individual that is within the optimal pro-inflammatory protein value range is indicative of good pro-inflammatory immune health and wherein a pro-inflammatory protein value that is outside the optimal pro-inflammatory protein value range is indicative of inferior pro-inflammatory immune health.

As discussed above, the methods according to the invention provide a method of assessing the pro-inflammatory immune health of a healthy individual. The first step in the method is to establish a benchmark pro-inflammatory protein value set for levels of pro-inflammatory protein in non-blood body fluid of healthy individuals. The term “establish” is intended to include the reference to a previously established benchmark pro-inflammatory protein value set.

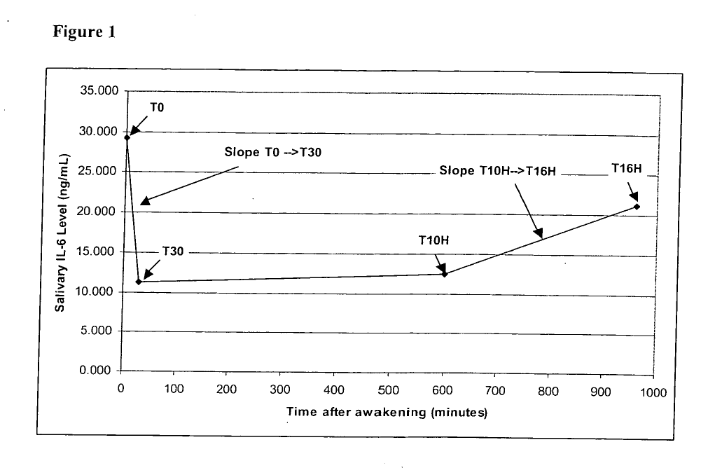

To establish the benchmark pro-inflammatory protein value set levels of pro-inflammatory protein in non-blood body fluid of healthy, individuals at least at one time point in a 24 hour period are measured. The individual is instructed to collect a number of non-blood body fluid samples throughout the day at prescribed times, preferably, relative to morning waking. These times should be selected in order to collect a non-blood body fluid, e.g., saliva, sample which will give the most accurate representation of a individual's pro-inflammatory protein levels and changes across the wakeful period of a 24 hour day. As discussed above, the term “pro-inflammatory protein value” means the result of a calculation made using levels of pro-inflammatory protein measured in samples of non-blood body fluid collected from healthy individuals at one or more time points in a 24 hour period, wherein the calculation could be as simple as measuring the level of pro-inflammatory protein in non-blood body fluid of samples collected at one time point in a 24 hour period. In another embodiment, the calculation could also be the calculation of the slope over time of the levels of pro-inflammatory protein as measured at least at two time points. FIG. 1 is a representation of a salivary IL-6 provile illustrating important time points for saliva collection. The profile is established by collected and analyzing samples of saliva at one or more time points relative to morning waking. As shown in the drawing, the time points of particular interest are at waking (T0), 30 minutes following waking (T30), 10 hours after waking (T10H), and 16 hours after waking (T16h).

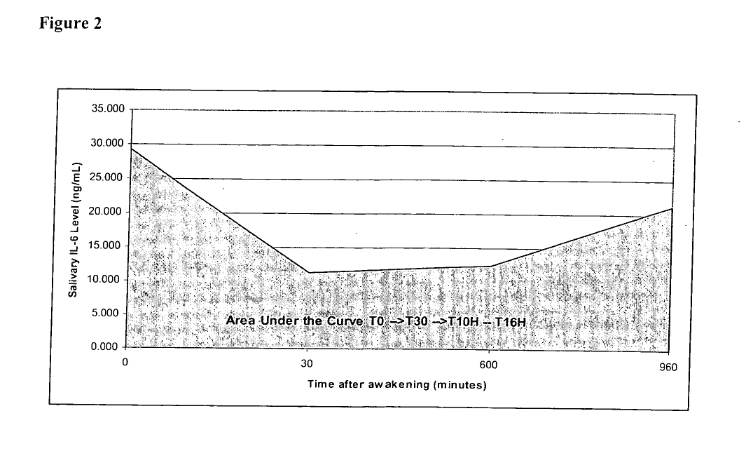

The calculation could also be the calculation of the area under the curve defined by the levels of pro-inflammatory protein in non-blood body fluid at least at two time points. The area under the curve is a particularly useful calculated value because it is representative of the total amount of secretion throughout a 16 hour period of the day. FIG. 2 illustrates the area under the curve defined by the level of pro-inflammatory protein, in this case interleukin-6, at time of waking (T0), 30 minutes after waking (T30), 10 hours after waking (T10H), and 16 hours after waking (T16H). The area under the curve is represented by the shaded area.

In a preferred embodiment, the level of pro-inflammatory protein is measured at least at one time point selected from (1) at or about waking; (2) about 30 minutes post waking; (3) about 60 minutes post waking; (4) about 6 hours post waking; (5) about 10 hours post waking and (6) about 16 hours post waking. In order to achieve the most reliable and accurate secretory profile, pro-inflammatory protein values are measured at each of the aforementioned time points. However, it has been discovered that meaningful pro-inflammatory protein measures can be ascertained when the value of pro-inflammatory protein is measured at or about waking; about 30 minutes post waking; about 10 hours post waking and about 16 hours post waking.

One method of determining the value of pro-inflammatory protein in non-blood body fluid is to measure the level of pro-inflammatory protein at least at two time points in a 24 hour period. The two time points can be selected from (1) at or about waking; (2) about 30 minutes post waking; (3) about 60 minutes post waking; (4) about 6 hours post waking; (5) about 10 hours post waking and (6) about 16 hours post waking. In a preferred embodiment, the level of pro-inflammatory protein is measured at least at two of the following time points: (1) at or about waking; and (2) about 30 minutes post waking; (3) about 10 hours post waking; and (4) about 16 hours post waking.

The value of pro-inflammatory protein can also be determined by calculating the area under the curve defined by the levels of the pro-inflammatory protein in non-blood body fluid of the individual as measured at least at two time points selected from: (1) at or about waking; (2) about 30 minutes post waking; (3) about 60 minutes post waking; (4) about 6 hours post waking; (5) about 10 hours post waking and (6) about 16 hours post waking versus the respective times. In another embodiment, the pro-inflammatory protein value is determined by calculating the slope over time of the levels of the pro-inflammatory protein in the non-blood body fluid of the individual as measured at least at two time points selected from: (1) at or about waking; (2) about 30 minutes post waking; (3) about 60 minutes post waking; (4) about 6 hours post waking; (5) about 10 hours post waking and (6) about 16 hours post waking. For example, the pro-inflammatory protein value can be determined by calculating the slope over time of the levels of the pro-inflammatory protein in the non-blood body fluid of the individual as measured at the time of waking and 30 minutes post waking. In yet another embodiment, the pro-inflammatory protein value is determined by calculating the slope over time of the levels of the pro-inflammatory protein in the non-blood body fluid of the individual as measured at about 10 hours post waking and about 16 hours post waking. In another embodiment, the pro-inflammatory protein value is determined by calculating the ratio of the level of pro-inflammatory protein in the non-blood body fluid as measured at or about waking and the level of pro-inflammatory protein in the non-blood body fluid as measured at about 16 hours post waking.

In one embodiment of the present invention, the non-blood body fluid referenced above, includes, for example, saliva, nasal secretions, tears, breath, semen, or any combination thereof. These fluids are typically collected in a collecting container. For example, a subject is asked to expectorate or allow his saliva to flow into an appropriately placed collecting container, such as a tube. An example of appropriate tubes are the 10 mL non-sterile and sterile tubes available from Sarstedt, Inc., of Newton, N.C., including, for example, article nos. 60.9924.283 and 62.9924.284, respectively. It is important to select materials for collection of the fluid that do not interfere with the pro-inflammatory protein, e.g., do not bind to the protein. For example, dental cotton should not be used because the protein is likely to bind to it. Stimulants of salivary secretion, such as a stick of chewing gum or crystalline stimulant, such as a citric drink mix or tart candy, may be used to enhance the flow of saliva. The collection of fluid samples could also be completed using a kit as described in detail below.

As defined above, the benchmark pro-inflammatory protein value set is a range of the level of pro-inflammatory protein in a non-blood body fluid taken from a number of healthy individuals sufficient to establish a normal distribution of the pro-inflammatory protein levels of the individuals. As defined above, “normal distribution” means a distribution of the density of a set of values that is approximately symmetric and has an approximately bell-shaped density curve with a single peak, as tested by fulfilling the requirements of a normality test. The normal distribution of a given data set can be tested using standard normality tests well known to those skilled in the art. Suitable normality tests useful in the present invention include the Anderson-Darling test, the Ryan-Joiner test, and the Kolmogorov-Smimov test.

The number of individuals needed for a useful benchmark pro-inflammatory protein value set can vary as long as the number is sufficient to establish a normal distribution. In one embodiment, the benchmark pro-inflammatory protein value set is established by measuring the pro-inflammatory protein value of greater than about 25 healthy individuals, for example, about 45 healthy individuals.

Once the benchmark pro-inflammatory protein value set has been established, an optimal pro-inflammatory protein value range can be determined by using descriptive statistic methodology, i.e., any method designed to summarize the information in a data set. For example, the descriptive statistic methodology can be any of the following:
(a) calculating the median value and defining the optimal range as the median value +/−10%;
(b) calculating the median value and defining the optimal range as the median value +/−20%;
(c) calculating the median value and defining the optimal range as the mean value +/−10%;
(d) calculating the median value and defining the optimal range as the mean value +/−20%;
(e) calculating the median value and defining the range as the mean value +/−1 standard deviations;
(f) calculating the median value and defining the range as the mean value +/−2 standard deviations;
(g) dividing the data into 10th percentiles and define the optimal range as a subset of the 10th percentiles;
(h) dividing the data into 10th percentiles and define the optimal range as a subset of the 10th percentiles wherein the optimal range is defined as the 30th through 70th percentile; and
(i) dividing the data into 10th percentiles and define the optimal range as a subset of the 10th percentiles, wherein the optimal range is defined as the 40th through 60th percentile.

It is important to note that more than one optimal pro-inflammatory protein value range can be determined, for example, by varying levels of immune health, such as, for example, inferior, fair, very good, excellent.

The level of pro-inflammatory protein in non-blood body fluid at each time point can be measured using appropriate analytical techniques, including but not limited to, radio immuno-assay (“RIA” and enzyme-linked immuno sorbent assay (“ELISA”) methods. ELISA methodologies are particularly preferred because they are able to measure pro-inflammatory protein levels in saliva at very low levels. Once these levels are measured, they are used to calculate the pro-inflammatory protein values discussed above.

Once the benchmark pro-inflammatory protein value set has been established and the optimal pro-inflammatory protein value range is determined, an individual who desires an assessment of his or her pro-inflammatory immune health should collect a number of non-blood body fluid, e.g., saliva, samples at least at one time point in a 24 hour period. As discussed above, one advantage of the present invention is that the benchmark pro-inflammatory protein value set can consist of values determined from samples previously collected from individuals other than the individual being assessed. Generally, the sample should be taken from the individual to be assessed at the same time point as a sample taken in establishing a benchmark pro-inflammatory protein value set. It is important to note that the sample taken for the individual being assessed does not have to be taken on the same day as the samples taken to establish the benchmark pro-inflammatory protein value set. For example, when the benchmark pro-inflammatory protein value set is established by determining the value of pro-inflammatory protein (1) at or about waking; (2) about 30 minutes post waking; (3) about 60 minutes post waking; (4) about 6 hours post waking; (5) about 10 hours post waking and (6) about 16 hours post waking, the individual to be assessed should measure the value of pro-inflammatory protein at least at one of the aforementioned time points, but can do so on a different day even a different year as the benchmark data set establishment. Another example would be that when the benchmark pro-inflammatory protein value set is established by determining the value of pro-inflammatory protein at waking and 30 minutes post waking, the individual to be assessed should measure the value of pro-inflammatory protein at either at waking or 30 minutes post waking or both.

The value of pro-inflammatory protein is calculated as described above. In other words, the calculation could be as simple as measuring the level of pro-inflammatory protein in non-blood body fluid. The calculation could also be the calculation of the area under the curve defined by the levels of pro-inflammatory protein in non-blood body fluid at least at two time points. In another embodiment, the calculation could also be the calculation of the slope over time of the levels of pro-inflammatory protein as measured at least at two time points. In one embodiment of the invention, the calculation is completed by calculating the curve defined by the levels of pro-inflammatory protein as measured at least at two of the following times: (1) at waking; (2) 30 minutes post waking; (3) 60 minutes post waking; (4) 6 hours post waking; (5) 10 hours post waking and (6) 16 hours post waking versus the respective times as described above. Further, the calculation could be calculating the slope over time of the levels of pro-inflammatory protein as measured at least at two of the following times: (1) at waking; (2) 30 minutes post waking; (3) 60 minutes post waking; (4) 6 hours post waking; (5) 10 hours post waking and (6) 16 hours post waking versus the respective times as described above.

Once the value of pro-inflammatory protein of the individual to be assessed is calculated, this value is then compared to the optimal pro-inflammatory protein value range described above. When the pro-inflammatory protein value of the individual being assessed is within the optimal pro-inflammatory protein value range, it is indicative of good pro-inflammatory immune health. On the other hand, when the pro-inflammatory protein value of the individual being assessed is outside the optimal pro-inflammatory protein value range, it is indicative of inferior pro-inflammatory immune health. In situations where inferior pro-inflammatory immune health is indicated, a program to improve pro-inflammatory immune health suitable programs, as described below can be recommended to the individual. Suitable programs for improving pro-inflammatory immune health are described below.

The comparison of the value of pro-inflammatory protein of the individual to be assessed to the optimal pro-inflammatory value range, gives the individual an objective measure of their pro-inflammatory protein level.

As discussed above, numerous lifestyle factors such as, stress, high body mass index, lack of sleep, smoking, caffeine consumption, and alcohol consumption have been implicated in causing chronic, low-grade inflammation. Accordingly, in yet another embodiment, the method of assessing the pro-inflammation immune health of a healthy individual of the invention further comprises a step for assessing the effect of lifestyle factors on pro-inflammatory health.

Relevant lifestyle factors for consideration and assessment include but are not limited to the quantity and/or frequency of smoking or chewing any form of tobacco, sleep quantity and quality, alcohol consumption, caffeine consumption, body weight, and stress, both chronic and acute. Numerous methods exist for measuring or obtaining information relating to these lifestyle factors including self-report questionnaires, interviews, psychometric assessment tools and biometric assessment tools. Medical information can be used such as, for example, height, weight, sleep behaviors, sleep quantity, perceived stress level, typical amount of caffeine consumed, typical amount of daily exercise, whether or not the subject has children, and a list of any medication being used.

Information regarding lifestyle factors may be subjective information or objective information. Subjective information may be obtained from the individual by questioning means, that is, by having the individual answer questions, which are asked of him or her, either orally or in written form, or electronically, such as via a computer terminal or other electronic device. The questioning means may be an interviewer asking oral questions of the individual, a written questionnaire on which the individual writes answers to the written questions, or an electronic questionnaire viewed by the individual on a computer screen or other electronic video device and for which the individual submits answers to the questions by typing on a keyboard, touching a responsive screen, speaking an answer, or the like.

Other personal information may be objective information, that is, it may be obtained by measuring certain properties or qualities, such as, for example, by taking physical or biological or other objective measurements, including, for example, alcohol levels, nicotine levels, caffeine levels in the blood or urine.

After the personal information is collected from the individual, it is used to create an individualized pro-inflammatory immune health treatment program for the individual. The information may be used to generate scores according to predetermined rules, formulae or algorithms, and these scores used in the selection of the elements of the individualized pro-inflammatory immune health program.

For smoking, the assessment could involve asking the individual questions such as, “Do you smoke?”, “How long have you been smoking?”, “How much do you smoke?”. Similar questions such as “How much coffee or alcohol do you consumer daily?” could be used to assess caffeine or alcohol consumption. Biometric assessment methodologies also exist for alcohol and are detailed in Biochemical markers of alcohol consumption Alcohol Health & Research World, Fall, 1990 by Alan S. Rosman, Charles S. Lieber which is hereby incorporated by reference.

Sleep quantity and quality can be assessed using simple questions, like “How long do you typically sleep at night?” or “Do you have any difficulties sleeping?”. However, it is preferable to use one of the many available sleep psychometric tools for assessing sleep including but not limited to The St. Mary's Hospital Sleep Questionnaire (Sleep. 1988 October;11(5):448-53.) and the Leeds Sleep Evaluation Questionnaire (Hum Psychopharmacol. 2003 December;18(8):603-10.). Biometric assessments such as actigraphy and polysomnography are also useful in assessing sleep.

Body weight can be easily assessed by simply asking the individual their weight. Body mass index is an even more useful assessment of weight since it considers the individual's height. Detailed guidelines for calculating and using an individual's body mass index can be found, for example, in Nutr Metab Cardiovasc Dis. 2005 August; 15(4):310-5. which is hereby incorporated by reference. In addition to body mass index, it is also useful to assess and individual's waist circumference or waist to hip ratio. Details on these methods can be found, for example, in Nutr Metab Cardiovasc Dis. 2005 August; 15(4):310-5. which is hereby incorporated by reference.

Stress information can be collected psychologically using surveys, questionnaires, validated psychometric tools or any other method providing information relating to a person's degree of stress. For example, a questionnaire that simply asks an individual to record how much stress he experiences is one suitable method of collecting stress information. In a preferred embodiment, stress information is collected using a stress psychometric tool. A detailed description of many suitable stress psychometric tools can be found in Cohen S, Kessler K and Underwood Gordon L. Measuring Stress: A guide for Health & Social Scientists, New York: Oxford University Press, 1997, Chapters 4-7, the disclosure of which is incorporated herein by reference. Examples of suitable stress psychometrics include the Trier Inventory of Chronic Stress, version 1 (Schulz, P. and Schlotz, W. Diagnostica, 1999; 45: 8-19.); the Trier Inventory of Chronic Stress, Version 2 (TICS 2), Schulz, P., & Schlotz, W. (2002) [Das Trierer Inventar zur Erfassung von chronischem Stress—Version 2 (TICS 2)], Trierer Psychologische Berichte, Band 29, Heft 2. Trier: Universität, Fachbereich I—Psychologie); the Perceived Stress Scale (Cohen, S. Kamarck, T. and Mermelstein, R. A global measure of perceived stress. Journal of Health & Social Behavior, 1983; 24: 385-396.); and the State-Trait Anxiety Inventory (Stait-Trait Anxiety Inventory: Spielberger, C D. and Vagg, P R. J. Pers. Assess. 1984, February; 48(1): 95-97), the disclosures of which are hereby incorporated by reference.

In a preferred embodiment, stress information is collected using the Trier Inventory of Chronic Stress, version 2, (TICS-2). The TICS-2 is a 62-item questionnaire that assesses various dimensions of an individual's chronic stress. The eleven dimensions in the TICS-2 are Work overload, Performance pressure at work, Social responsibility, Performance pressure in social situations, Overextended at work, Social isolation, Social failure, Worry propensity, Aversive work load, Fatigue, and Social conflicts. The TICS-2 is particularly useful in collecting stress information because it shows the relative importance of multiple dimensions of stress in the individual's life. Other psychometric tools that similarly assess multiple dimensions of stress would also be particularly useful and are deemed to be within the scope of this invention.

Additionally, there are a number of physiological ways to collect stress information. Physiological means of gathering stress information include biometric tools such as skin conductance, heart rate, heart rate variability, blood pressure, skin temperature or assessment of stress-related biomarkers in blood or saliva. Diffuse Reflectance Spectroscopy (DRS), as described in copending application Ser. No. 10/353,525, filed Jan. 29, 2003 entitled “Method Of Measuring The Stress Or Relaxation Level Of A Mammal”, the disclosure of which is hereby incorporated by reference, is another physiological way of collecting stress information. Chronic stress, the accumulated stress over time, is most important in influencing an individual's acne condition. A preferred method of measuring chronic stress is the objective, non-invasive method of using salivary cortisol levels described in detail in copending U.S. patent application Ser. Nos. 10/012,627 filed Dec. 7, 2001 entitled “Methods of Measuring Stress In Mammals” the disclosure of which is hereby incorporated by reference.

In another embodiment, the invention relates to a method of recommending a treatment program to improve or maintain pro-inflammatory immune health. The method comprises assessing the pro-inflammatory immune health of an individual as outlined above and then recommending a pro-inflammatory immune health treatment program. In one embodiment, the invention relates to a method for recommending a program to improve or maintain pro-inflammatory immune health of a healthy individual comprising:
(a) establishing a benchmark pro-inflammatory protein value set wherein the pro-inflammatory protein value is determined by a calculation made using levels of pro-inflammatory protein measured in samples of non-blood body fluid collected from healthy individuals at one or more time points in a 24 hour period;
(b) determining an optimal pro-inflammatory protein value range from the benchmark pro-inflammatory protein value set established in (a) by using descriptive statistic methodology;
(c) establishing a pro-inflammatory protein value for said individual to whom a program will be recommended, wherein said value is determined by the same calculation set forth in (a); and
(d) comparing the value established in step (c) to the optimal pro-inflammatory protein value range determined in step (b), wherein a pro-inflammatory protein value for said individual that is within the optimal pro-inflammatory protein value range is indicative of good pro-inflammatory immune health and wherein a pro-inflammatory protein value that is outside the optimal pro-inflammatory protein value range is indicative of inferior pro-inflammatory immune health;
(e) wherein when the pro-inflammatory protein value for said individual is within the optimal pro-inflammatory protein value range, a program that comprises instructions for the individual to continue practicing his or her current lifestyle factors is recommended;
(f) wherein when the pro-inflammatory protein value for said individual is outside the optimal pro-inflammatory protein value range, a program that comprises instructions for the individual to change current lifestyle factors is recommended.

The program that comprises instructions for the individual to change current lifestyle factors may include a recommendation to use certain products and services, participate in certain activities, gain education, participate in counseling or a combination thereof. Any method useful for improving the lifestyle, health, well-being and/or emotional state of an individual would be useful as a treatment program that addresses pro-inflammatory immune health. For example, methods for reducing chronic stress are useful such as the methods described in copending application Ser. No. 10/012,627, filed Dec. 7, 2001 entitled “Method For Reducing Chronic Stress in Mammals”, the disclosure of which is hereby incorporated by reference. Also useful are methods for improving sleep such as described in copending patent application Ser. No.: 10/357,648, filed Feb. 4, 2003 entitled “Method Of Affecting Sleep And Sleep-Related Behaviors”, the disclosure of which is hereby incorporated by reference.

In one embodiment, the program that comprises instructions for the individual to change current lifestyle factors may include a recommendation to seek psychological, emotional and/or social counseling, start an exercise program, including, for example, running, sports, tai chi, yoga, pilates, practice behavioral modification techniques, relaxation techniques and/or breathing techniques, improve sleep quality, increase sleep quantity, reduce or cease the smoking or chewing of tobacco, reduce caffeine consumption, reduce alcohol consumption, lose weight, and/or start an in person and/or internet coaching program and any other activity that would improve the individual's lifestyle, health, well-being and emotional state, including reading a book or magazine, watching movies, taking a vacation, and meditation. Other examples include the administration of herbal supplements, vitamins or special food, and/or over the counter or prescription medications. Examples of coaching programs can be found in Diabetes Educ. 2004 September-October; 30(5):795-804, A Nurse-Coaching Intervention For Women With Type 2 Diabetes; Whittemore R, Melkus G D, Sullivan A, Grey M., the disclosure of which is hereby incorporated by reference.

One preferred program that comprises instructions for the individual to change current lifestyle factors would be a recommendation for the individual to participate in a health management program such as Weight Watchers. Suitable health management programs could be delivered in person, by phone, in print or through the internet. Programs could be designed for improving general health or could have a focus on a specific lifestyle factor such as sleep, weight loss, stress management, nutrition, alcohol consumption, caffeine consumption, or smoking cessation. The health management program may involve tailored information, coaching, motivational interviewing or social support, all of which are particularly useful in motivating an individual towards making behavioral change.

In one embodiment, the recommended programs described above are delivered via a personalized report which includes age, name, gender, product preferences, family composition, address, coupons, a summary of personal lifestyle factors or a combination thereof.

As described above, the method according to the invention provides a pro-inflammatory immune health assessment tool which is reliable and sensitive. It would also be useful to provide a tool to assess the efficacy of a program to improve or maintain pro-inflammatory immune health, including, for example the recommendations and programs to change current lifestyle factors described above. Such an assessment would provide important information regarding the effectiveness of the program which would help the individual to determine whether or not to continue with his or her current program or revise the program. The assessment would also be useful to companies seeking to evaluate the efficacy of newly developed programs to improve or maintain pro-inflammatory immune health, i.e., for claim support.

Accordingly, in another embodiment, the invention relates to a method for assessing the efficacy of a program to improve or maintain pro-inflammatory immune health of a healthy individual. The method comprises:
(a) prior to the start of said program, assessing the baseline pro-inflammatory immune health of said individual by:
   (i) establishing a benchmark pro-inflammatory protein value set wherein the pro-inflammatory protein value is determined by a calculation made using levels of pro-inflammatory protein measured in samples of non-blood body fluid collected from healthy individuals at one or more time points in a 24 hour period;
   (ii) determining an optimal pro-inflammatory protein value range from the benchmark pro-inflammatory protein value set established in (i) by using descriptive statistic methodology;
   (iii) establishing a pro-inflammatory protein value for said individual for whom the efficacy of a program to improve pro-inflammatory immune health is being assessed, wherein said value is determined by the same calculation set forth in (i); and
   (iv) comparing the value established in step (iii) to the optimal pro-inflammatory protein value range determined in step (ii), wherein a pro-inflammatory protein value for said individual that is within the optimal pro-inflammatory protein value range is indicative of good pro-inflammatory immune health and wherein a pro-inflammatory protein value that is outside the optimal pro-inflammatory protein value range is indicative of inferior pro-inflammatory immune health.
(b) after the start of said program to improve or maintain pro-inflammatory immune health of a healthy individual, assessing the post-program pro-inflammatory immune health of said individual according to steps (i) to (iv) set forth in (a);
(c) comparing the post-program pro-inflammatory immune health assessed in step (b) to the baseline pro-inflammatory immune health assessed in step (a);
(d) wherein a change from inferior pro-inflammatory immune health to good pro-inflammatory immune health is an indication that the program is efficacious.

As discussed above, chronic increase in the basal level of pro-inflammatory activity effects immune health and has been linked to numerous health conditions such as cancer, heart disease, diabetes, chronic skin disorders, asthma, allergy, arthritis, mental/behavioral illness, auto-immune disorders (lupus, MS, etc.) It is also recognized that people caught up in the hectic lifestyles, often ignore health issues raised by their current lifestyle factors. Accordingly, it would be beneficial to provide a method for motivating a healthy individual to improve lifestyle factors in order to improve pro-inflammatory immune health. It is believed that if the individual recognizes or can actually and easily see the effect their lifestyle factors have on pro-inflammatory immune health the individual would be more motivated to change lifestyle factors. Because the above described method of assessing pro-inflammatory immune health provides an accurate, reliable and sensitive assessment it provides such a tool.

Accordingly, in another embodiment, the invention relates to a method of motivating a healthy individual to improve lifestyle factors. The method comprises:
(a) assessing the pro-inflammatory immune health of a healthy individual comprising:
   (i) establishing a benchmark pro-inflammatory protein value set wherein said pro-inflammatory protein value is determine by a calculation made using levels of pro-inflammatory protein measured in samples of non-blood body fluid collected from healthy individuals at two or more time points in a 24 hour period;
   (ii) determining an optimal pro-inflammatory protein value range from the benchmark pro-inflammatory protein value set established in (i) by using descriptive statistic methodology;
   (iii)establishing a pro-inflammatory protein value for said individual to be assessed, wherein said value is determined by the same calculation as set forth in (i); and
   (iv)comparing the value established in step (iii) to the optimal pro-inflammatory protein value determined in step (ii), wherein a pro-inflammatory protein value for said individual that is within the optimal pro-inflammatory protein value range is indicative of good pro-inflammatory immune health and wherein a pro-inflammatory protein value that is outside the optimal pro-inflammatory protein value range is indicative of inferior pro-inflammatory immune health;
   (b) reporting comparison of (iv) to said individual; wherein said individual is motivated by said comparison to improve, change, or maintain lifestyle factors.

In another embodiment, the comparison step in the method for motivating is completed periodically throughout a recommended treatment program, such as, for example, the treatment programs described above.

The reporting step can be delivered to the individual by various report means. For example, the report could be delivered to the individual during counseling sessions or motivational interviewing techniques. An example of a motivational interviewing technique includes those described in Br J Gen Pract. 2005 April;55(513):305-12, Motivational interviewing: a systematic review and meta-analysis. Rubak S, Sandbaek A, Lauritzen T, Christensen B, the disclosure of which is hereby incorporated by reference .

In another embodiment, the report can be delivered via the internet, including email and web sites. In cases where the report is delivered via a website, the website could beneficially include a point of sale for treatment products and spa services which provide pro-inflammatory immune health benefits. Such products and/or spa services could include, for example, the recommendations to change current lifestyle factors and the treatment programs that address pro-inflammatory immune health described above.

The report could also be conveniently delivered through a retail shopping environment. By “retail shopping environment” it is meant business establishments stocked with items for sale where consumers go to examine goods and services with the possible intent to buy for their personal or household use. Examples of such retail shopping environments include, without limitation, department stores, shopping malls, shopping centers, kiosks, drug stores, mass merchandisers, specialty shops, grocery stores, and convenience stores. Typically, consumers may come and go freely during the normal operating business hours of these retail shopping environments. In other words, customers do not need to make appointments to visit such retail shopping environments.

In another embodiment, the report can be delivered through direct to consumer channels, such as through telemarketing, personal selling, e.g., door to door, infomercials, home shopping channels, such as, QVC. The report could also be delivered as part of treatment products and spa services, for example, as part of treatment products and spa services which provide pro-inflammatory immune health benefits, including, for example, the recommendations to change current lifestyle factors and the treatment programs that address pro-inflammatory immune health described above.

The report may include the level of pro-inflammatory protein in each of the said individual's non-blood body fluid samples and could also include the calculated values of pro-inflammatory protein as previously described. The report is of particular use if it includes an illustration of the comparison of the individual's pro-inflammatory protein values to the optimal pro-inflammatory protein range. This comparison could be illustrated in a table, graph, pie chart or other illustrative format.

The method of assessing pro-inflammatory immune health described herein enables the individual to assess their pro-inflammatory immune health without the need for consultation with a medical professional to administer testing and interpret results. It would therefore be beneficial to provide the individual with a kit that contains the required materials to collect the non-blood body fluid as described above. Accordingly, the present invention also provides test kits for use with the methods of the present invention. The kit of the present invention is useful for measuring pro-inflammatory protein values in a non-blood body fluid. Each kit contains detailed instructions on the collection of the non-blood body and information on where to send the resulting collection.

An example of a kit for measuring pro-inflammatory immune health according to the invention, comprises:
a.) at least two saliva sample collection tubes; and
b.) instructions on how to collect, store and ship saliva samples, wherein said instructions require the collection of saliva samples at least one time point relative to morning waking.

The time points relative to morning waking include those previously discussed such as, for example, at or about waking, about 30 minutes post waking, about 60 minutes post waking, about 6 hours post waking, about 10 hours post waking and about 16 hours post waking.

The kit could also include a log sheet or card to record sample collections times as well as an ice pack.

In one embodiment, the kit can advantageously further comprise a website link, user ID and password for use in accessing results. The website can be as described above, i.e., which could beneficially include a point of sale for treatment products and spa services which provide pro-inflammatory immune health benefits, including, for example, the recommendations to change current lifestyle factors and the treatment programs that address pro-inflammatory immune health described above.

The kit may be sold for example through a retail shopping environment, on the internet, through direct to consumer channels each described above in detail. The kit could also be sold as part of treatment products and spa services, for example, as part of treatment products and spa services which provide pro-inflammatory immune health benefits as described in detail above. In certain cases, the cost of said kit may be beneficially included in the price of one or more treatment products and spa services discussed above.

The sample collection tubes provided in the kit may contain an absorbent material for the collection of non-blood body fluid as discussed previously.

The kit may also include the questionnaires described above as useful for assessing lifestyle factors information. The questionnaires could be provided by paper questionnaires or by a link to assess the questionnaire on the Internet.

In order to illustrate the invention the following examples are included. These examples do not limit the invention. They are meant only to suggest a method of practicing the invention.

### EXAMPLES

### Example I

Establishing A Benchmark Pro-Inflammatory Protein Value Set

45 healthy, male and post-menopausal female human subjects were recruited to participate in a study to investigate the diurnal rhythm and levels of IL-6 in saliva. In order to qualify as healthy, subjects had to self-identify as not having any chronic, immune-related medical conditions including autoimmune disorders, chronic illness, or other self-identified chronic condition. Medical information was collected from the subjects including height, weight, sleep behaviors, perceived stress level, typical amount of caffeine consumed, typical amount of daily exercise, whether or not the subject has children, and a list of any medication being used. Sleep behaviors were tracked using the St. Mary's Sleep Questionnaire. Perceived stress was assessed using the Perceived Stress Scale (PSS) as well as the Trier Inventory of Chronic Stress, version 2 (TICS2).

Each subject was provided with a kit to use to collect saliva samples. The kit was comprised of a plastic bag containing labeled plastic tubes, a card to use for recording sample collection times, absorbent material, and saliva collection instructions. The subjects collected saliva samples on four days, two days during the first week (Days 1 and 2) and two days during the following week (Days 3 and 4), for a total of 180 days of samples, i.e. 45 subjects with 4 days of samples each. Subjects collected saliva six times throughout the day, including upon morning waking (T0), 30 minutes after waking (T30), 60 minutes after waking (T60), 6 hours after waking (T6H), 10 hours after waking (T10H), and 16 hours after waking (T16H). Subjects were asked to passively drool into the tube for approximately 2 minutes or until at least 1.5 ml of saliva had been collected. The samples were immediately frozen in a standard household freezer and were subsequently stored at −70 degrees C. The level of IL-6 in each sample was determined using a standard enzyme linked immunoassay (ELISA) specific to IL-6. Samples were tested in duplicate, and the average of the two results was used for all analyses. Prior to data analyses, several high values were recoded to three standard deviations above the mean (separate by time of day). For T0, 7 subjects who had IL-6 values between 87 and 104 were recoded to 84; at T30, 3 subjects had IL-6 values between 41 and 100 and were recoded to 39.6. Six subjects with IL-6 values from 40 to 54 were recoded to 39.6 and at T6h, 4 subjects with IL-6 values between 56 and 100 were recoded to 54.6. At T10h, six subjects with IL-6 values from 53 to 69 were recoded to 48.14. At T16h, six subjects with IL-6 values ranging from 84 to 112 were recoded to 83.01. The data were sufficiently positively skewed, range 1.93 to 2.265, to warrant a log transformation; after transformation the data were normally distributed, skew range 0.306 to 0.892.

To evaluate whether IL-6 levels are the same across five days, we used a 4(day)×6(time of day) repeated measures ANOVA. There was a significant main effect of time of day, F(5, 435)=11.845, p<0.001, no main effect of day, F(3, 87)=0.507, p =0.678, and no interaction between time and day, F(15, 435)=0.501, p=0.940. We also used a 2(day)×6 (time of day) repeated measures ANOVA to evaluate the diurnal rhythm of IL-6 in subjects with data one week apart. There was a significant main effect of time of day F(5, 270)=14.852, p<0.001, no main effect of day, F(1, 54)=0.155, p=0.696, and a trend for an interaction between day and time, F(5, 270)=2.21 1, p=0.053. Follow-up paired samples t-tests revealed no time for which IL-6 levels were different across days, ts range −1.518 to 1.111, ps>0.132. Thus, it is possible to conclude that IL-6 levels are not significantly different from each other across days.

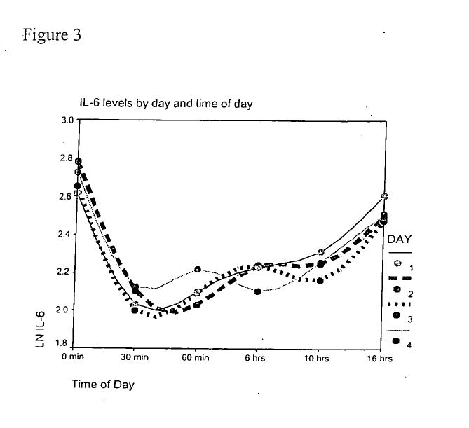

The diurnal rhythm of salivary IL-6 is not primarily linear. As evidenced by our data and seen in FIG. 3, IL-6 levels vary across the day in an interesting pattern: IL-6 levels are high immediately at waking, sharply decline by 30 minutes later, remain low for the early part of the day, then steadily increase across the day and reach a late peak in levels 16 hours after waking. Paired samples t-tests reveal that IL-6 levels are significantly different from each other at all points in the day, ps<0.001; however all time points are significantly correlated with each other, rs range 0.294 to 0.671, ps<0.001.

To evaluate whether the diurnal rhythm of IL-6 is the same across days, we examined the proportion of the total variability in IL-6 that is accounted for by linear, quadratic, cubic and 4^{th} order polynomial variation (eta²). When collapsed across days, there is a significant quadratic, cubic and 4^{th} order polynomial trend. The quadratic and 4^{th} order functions are consistent across days but the cubic trend is only evident when collapsed across all five days. The diurnal rhythm displays quadratic, cubic and 4^{th} order polynomial trends. The shape of the diurnal rhythm is consistent across days.

The shape of the diurnal rhythm is consistent across individuals though much variability in IL-6 levels exists. To evaluate individual differences, we divided subjects into two clusters based on their IL-6 levels across time. The first cluster consists of subjects with low IL-6 levels and the second cluster includes subjects with high IL-6 levels across the day. We then examined the diurnal rhythm in both clusters (collapsed across days) and found that both clusters have significant quadratic, cubic and 4^{th} order polynomial trends. Thus, the diurnal rhythm appears consistent across individuals.

To determine the number of samples needed to estimate the diurnal rhythm accurately, we used the eta² presented in Table 1. This analysis is collapsed across days and considers models with both the highest eta² and the most optimal fit to the model with six measures. When five IL-6 measures are available, the highest eta², 0.637, is obtained by sampling at T0 T30 T60 T6h and T10h. However, the size of the eta² is largely driven by the linear function which does not appear in the six sample model. Sampling at T0 T30 T6h T10h and T16h returns a model with a high eta², particularly for the 4^{th} order and cubic functions. When the quadratic and cubic functions are of interest, including samples at T0 T30 T60 T10h and T16h may be optimal. The curve appears to be optimally estimated—preserving the quadratic, cubic and 4^{th} order trend—using samples collected at T0, T30, T10h, T16h and either T60 or T6h depending on whether the 4^{th} order or the quadratic function is of interest. When four samples are available, sampling at T0 T30 T60 and T6h or T0 T30 T60 and T10h returns the highest eta² values. However, when the quadratic curve is of interest, sampling at T0 T30 T60 and T16h may be optimal. When both the quadratic and cubic curves are of interest, sampling at T0 T30 T6h and T16h appears best. Sampling at T0 T30 T6h and T16h is recommended in order to best approximate the six sample model when four samples are available. The curve can be estimated using T0 T30 T60 or T0 T30 T10h to obtain the highest eta² value when three samples are available. However, the shape of the diurnal rhythm is better approximated using T0 T30 and T16h or T0 T60 and T16h when three measures are available.

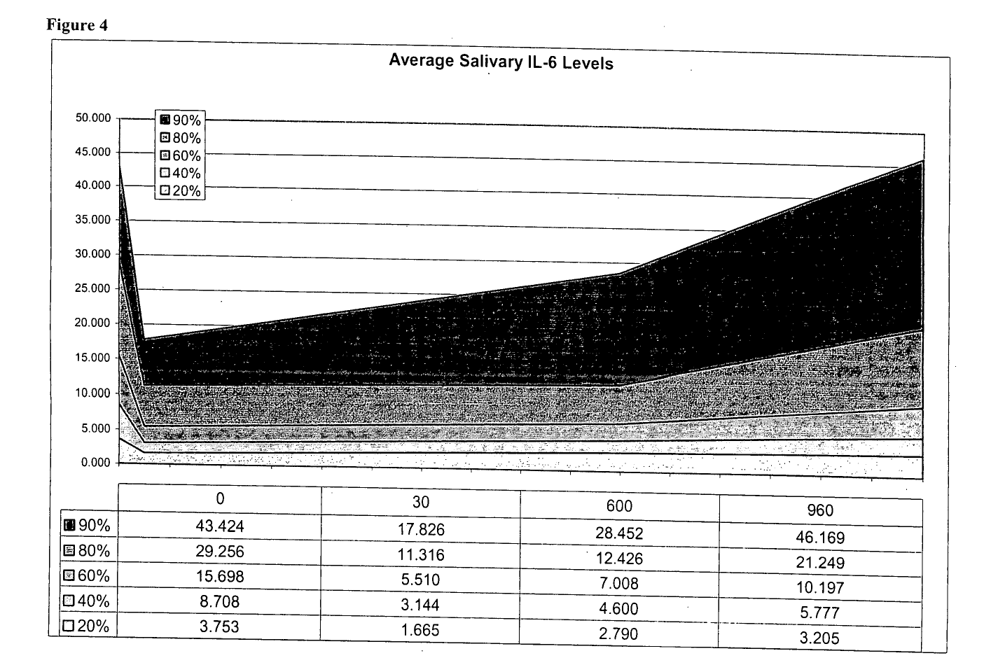

Ranges for IL-6 levels across the six times of day are presented in FIG. 4 below. As also shown in FIG. 4, 95% confidence intervals were calculated separately for reference values for the each consecutive 10^{th} percentile score.

To assess whether behavioral associations with IL-6 would be reliably detected with the size of the current dataset, we examined power for expected effect sizes in the range typically observed between hormones and behavior (correlations from 0.10 to 0.30). With 180 subjects, we would accurately reject the null hypothesis 27% of the time with a population correlation of 0.10. We would need to increase the sample size to approximately 800 to 900 subjects to reliably detect behavioral associations that are this small. With an expected correlation of 0.20, however, we have adequate power (a 77% chance of accurately rejecting the null hypothesis) with the current sample. With an expected correlation of 0.30, we would have a 98% chance of accurately rejecting the null hypothesis. Thus, we would conclude that the current sample size is adequate to detect expected correlations above 0.20. There does not appear to be any need to sample additional subjects.

Several conclusions can be reached from this set of analysis. It appears that IL-6 levels vary across the day in an interesting pattern: IL-6 levels are high immediately at waking, sharply decline by 30 minutes later, remain low for the early part of the day, then steadily increase across the day and reach a late peak in levels 16 hours after waking. The shape of the diurnal rhythm appears largely quadratic and 4^{th} order function and, to a lesser extent, cubic. The diurnal rhythm of IL-6 appears to be optimally estimated using three samples if the quadratic trend is of primary interest, but five samples may be needed if the cubic and 4^{th} order functions are of interest. Variance in IL-6 appears to be accurately captured by two to three days of data collection. IL-6 levels appear consistent across days, but the shape of the diurnal rhythm is not. When interested in group differences (between-subjects analyses), the diurnal rhythm of IL-6 does not appear different from one day to the next so multiple days of data collection may not be necessary. However, when interested in individual differences in the diurnal rhythm of IL-6 (within-subjects analyses), the diurnal rhythm of IL-6 is not consistent across days. Many days of sample collection may be needed before a stable rhythm emerges. A dataset of this size (N=180) is more than adequate to estimate the diurnal rhythm of IL-6 and associations with behavior can be reliably detected at correlations of 0.20 or greater.

Table 1 set forth below demonstrates the Eta-squared, the proportion of the total variability in the dependent variable that is accounted for by variation in the independent variable, for polynomial transformations of the diurnal rhythm described above.

TABLE 1

| | Linear | Quadratic | Cubic | 4^{th} Order | Total |
|---|---|---|---|---|---|
| Six IL-6 | | | | | |
| Measures | | | | | |
| T0, 30, 60, | 0.002 | 0.289 | 0.052 | 0.112 | 0.455 |
| 6 h, 10 h, 16 h | | | | | |
| Five IL-6 | | | | | |
| Measures | | | | | |
| T30, 60, 6 h, | 0.197 | 0.061 | 0.040 | 0.033 | 0.331 |
| 10 h, 16 h | | | | | |
| T0, 60, 6 h, | 0.008 | 0.282 | 0.063 | 0.117 | 0.470 |
| 10 h, 16 h | | | | | |
| T0, 30, 6 h, | 0.006 | 0.292 | 0.074 | 0.122 | 0.494 |
| 10 h, 16 h** | | | | | |
| T0, 30, 60, | 0.006 | 0.331 | 0.074 | 0.070 | 0.481 |
| 10 h, 16 h** | | | | | |
| T0, 30, 60, | 0.007 | 0.335 | 0.056 | 0.083 | 0.481 |
| 6 h, 16 h | | | | | |
| T0, 30, 60, | 0.131 | 0.282 | 0.190 | 0.034 | 0.637 |
| 6 h, 10 h* | | | | | |
| Four IL-6 | | | | | |
| Measures | | | | | |
| T0, 30, 60, | 0.186 | 0.262 | 0.152 | | 0.600 |
| 6 h* | | | | | |
| T0, 60, 6 h, | 0.158 | 0.248 | 0.149 | | 0.555 |
| 10 h | | | | | |
| T0, 6 h, 10 h, | 0.017 | 0.247 | 0.014 | | 0.278 |
| 16 h | | | | | |
| T0, 30, 6 h, | 0.150 | 0.267 | 0.152 | | 0.569 |
| 10 h | | | | | |
| T0, 30, 10 h, | 0.010 | 0.313 | 0.077 | | 0.400 |
| 16 h | | | | | |
| T0, 30, 60, | 0.174 | 0.284 | 0.140 | | 0.598 |
| 10 h* | | | | | |
| T0, 30, 60, | 0.017 | 0.343 | 0.024 | | 0.384 |
| 16 h** | | | | | |
| T0, 30, 6 h, | 0.011 | 0.321 | 0.056 | | 0.388 |
| 16 h** | | | | | |
| T0, 60, 6 h, | 0.013 | 0.305 | 0.048 | | 0.366 |
| 16 h | | | | | |
| T0, 60, 10 h, | 0.011 | 0.300 | 0.067 | | 0.378 |
| 16 h | | | | | |
| T30, 60, 6 h, | 0.056 | 0.000 | 0.010 | | 0.066 |
| 10 h | | | | | |
| T30, 6 h, 10 h, | 0.191 | 0.034 | 0.056 | | 0.281 |
| 16 h | | | | | |
| T30, 60, 6 h, | 0.196 | 0.090 | 0.012 | | 0.298 |
| 16 h | | | | | |
| T30, 60, 10 h, | 0.198 | 0.089 | 0.007 | | 0.294 |
| 16 h | | | | | |
| T60, 6 h, 10 h, | 0.167 | 0.047 | 0.053 | | 0.267 |
| 16 h | | | | | |
| Three IL-6 | | | | | |
| Measures | | | | | |
| T0, 30, 60* | 0.269 | 0.255 | | | 0.524 |
| T0, 60, 6 h | 0.198 | 0.244 | | | 0.442 |
| T0, 6 h, 10 h | 0.185 | 0.159 | | | 0.344 |
| T0, 10 h, 16 h | 0.019 | 0.224 | | | 0.243 |
| T0, 30, 6 h | 0.198 | 0.248 | | | 0.446 |
| T0, 30, 10 h* | 0.185 | 0.271 | | | 0.456 |
| T0, 30, | 0.019 | 0.336 | | | 0.355 |
| 16 h** | | | | | |
| T0, 60, 10 h | 0.185 | 0.263 | | | 0.448 |
| T0, 60, | 0.019 | 0.325 | | | 0.344 |
| 16 h** | | | | | |
| T0, 6 h, 16 h | 0.019 | 0.233 | | | 0.252 |
| T30, 60, 6 h | 0.036 | 0.010 | | | 0.046 |
| T30, 6 h, 10 h | 0.051 | 0.011 | | | 0.062 |
| T30, 10 h, | 0.193 | 0.027 | | | 0.220 |
| 16 h | | | | | |
| T30, 6 h, 16 h | 0.193 | 0.032 | | | 0.225 |
| T30, 60, 10 h | 0.051 | 0.015 | | | 0.066 |
| T30, 60, 16 h | 0.193 | 0.140 | | | 0.333 |
| T60, 10 h, | 0.174 | 0.038 | | | 0.212 |
| 16 h | | | | | |
| T60, 6 h, 16 h | 0.174 | 0.040 | | | 0.214 |
| T60, 6 h, 10 h | 0.035 | 0.008 | | | 0.043 |
| T6 h, 10 h, | 0.113 | 0.077 | | | 0.190 |
| 16 h | | | | | |

Numbers underlined are significant at p < 0.05.
NOTE:
*refers to the model(s) with the highest Eta-square.
**refers to the models which optimally maintain the shape of the diurnal rhythm.
For the purposes of these analyses, all data were collapsed across days.

### Example II

Measurement of Salivary IL-6 is Useful in Assessing the Impact of Lifestyle Factors on Pro-Inflammatory Health-Sleep

A group of 35 healthy, men and post-menopausal women participated in a study to investigate the influence of lifestyle factors on salivary IL-6 levels throughout the day. In order to qualify as healthy, subjects had to self-identify as not having any chronic, immune-related medical conditions including autoimmune disorders, chronic illness, or other self-identified chronic condition. Medical information was collected from the subjects including height, weight, sleep behaviors, perceived stress level, typical amount of caffeine consumed, typical amount of daily exercise, whether or not the subject has children, and a list of any medication being used.

Sleep behaviors were tracked using the St. Mary's Sleep Questionnaire (Sleep. 1988 October; 11(5): 448-53.). Perceived stress was assessed using the Perceived Stress Scale (PSS) (Cohen, S. Kamarck, T. and Mermelstein, R. A global measure of perceived stress. Journal of Health & Social Behavior, 1983; 24: 385-396.) as well as the Trier Inventory of Chronic Stress, version 2 (TICS2) (Schulz, P., & Schlotz, W. (2002) [Das Trierer Inventar zur Erfassung von chronischem Stress—Version 2 (TICS 2)], Trierer Psychologische Berichte, Band 29, Heft 2. Trier: Universitat, Fachbereich I—Psychologie). Each subject's body mass index was calculated according to standard methodology using the subject's self-reported height and weight information.

The subjects collected saliva samples on two days, two days during the first week (Days 1 and 2) and two days during the following week (Days 3 and 4). Subjects collected saliva six times throughout the day, including upon morning waking (T0), 30 minutes after waking (T30), 60 minutes after waking (T60), 6 hours after waking (T6H), 10 hours after waking (T10H), and 16 hours after waking (T16H). Saliva samples were collected in basic plastic tubes. Subjects were asked to passively drool into the tube for approximately 2 minutes or until at least 1.5 ml of saliva had been collected. The samples were immediately frozen in a standard household freezer and were subsequently stored at −70° C. The level of IL-6 in each sample was determined using a standard ELISA specific to IL-6. Samples were tested in duplicate, and the average of the two results was used for all analyses.

The St. Mary's Sleep Questionnaire is a well-known, validated psychometric tool that can be used in monitoring an individual's sleep on any given night. Specifically, the questionnaire assesses the following characteristics of sleep: depth of sleep, number of nighttime wakings, sleep quality, morning mood state (drowsy vs. alert), subject's satisfaction with sleep, latency to sleep, degree of difficulty falling asleep, and total amount of sleep.

Total sleep time was calculated for each subject in this study using responses to the St. Mary's Sleep Questionnaire. Subjects were characterized as having had “Low Sleep” if their total sleep time was less than 400 minutes (6 hours and 40 minutes) and characterized as having had “High Sleep” if their total sleep time was greater than 440 minutes (7 hours and 20 minutes). Subjects with sleep time between 400 and 440 minutes were excluded from analysis in order to minimize normal variability. A summary of the subjects total sleep time data is shown in Table 2 below.

TABLE 2
Sleep Time Versus Morning Salivary IL-6

| | | Total Sleep Time | Morning Salivary IL-6 |
|---|---|---|---|
| | Subject ID# | (minutes) | (ng*min/mol) |
| Low Sleep | 16 | 360 | 0.039 |
| (<401 mins) | 28 | 380 | 0.045 |
| | 10 | 400 | 0.037 |
| High Sleep | 18 | 440 | 0.009 |
| (>439 mins.) | 21 | 440 | 0.025 |
| | 19 | 455 | 0.018 |
| | 17 | 613 | 0.031 |

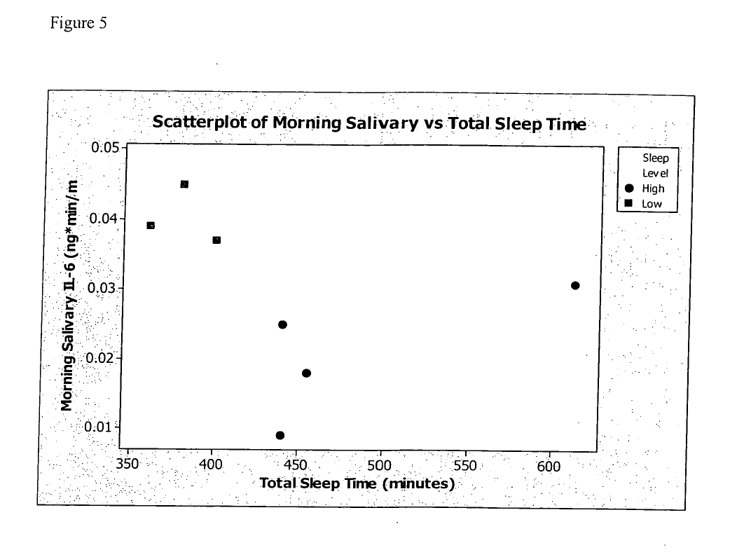

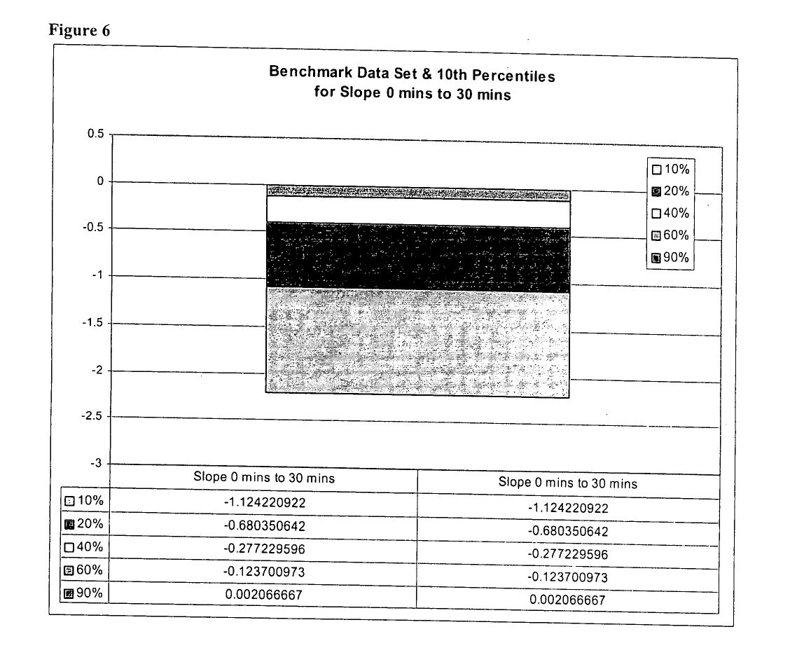

Morning salivary IL6 levels for the subjects are also shown in FIG. 5. where morning salivary IL6 was calculated as the area under the curve defined by salivary IL6 levels at T0 (morning waking), T30 (thirty minutes post waking) and T60 (sixty minutes post waking). Other cumulative values of morning IL-6, e.g. the area under the curve from T0 to T30, would be equally suitable.

A t-test on morning IL6 levels for the High Sleep vs. Low Sleep group indicated that there was a statistically significant difference in morning salivary IL-6 levels between the groups. Specifically, cumulative measures of morning salivary IL-6 levels were significantly lower by 48.6% in subjects in the High Sleep group vs. subjects in the Low Sleep. The scatter plot in FIG. 5 summarizes this finding. Thus, greater sleep time resulted in lower levels of IL-6 following morning waking. While not wishing to be bound by the theory, this effect of greater sleep leading to a lower levels of IL-6 following waking may be indicative of a more harmonious shift from Thl to Th2 with lower overall levels of circulating pro-inflammatory cytokines. In summary, the method according to the invention was shown to be useful in assessing the impact of lifestyle factors, in this case sleep time, on pro-inflammatory immune health.

### Working Example III

Using a Pre-Established Benchmark Pro-Inflammatory Protein Value Set

In one embodiment of the invention, the pro-inflammatory immune health of a healthy individual is assessed using a pre-established benchmark pro-inflammatory protein value set.

A benchmark pro-inflammatory protein value set of the values of salivary IL-6, a pro-inflammatory protein, is established by collecting the non-blood body fluid of 45 healthy, individuals as described in Example I.

Descriptive statistic methodology is used with the benchmark pro-inflammatory protein value set to determine an optimal pro-inflammatory protein value range. In this example, the data set is divided into 10^{th} percentiles, and, in one embodiment, the optimal value range is defined as the 40^{th} through 60^{th} percentiles. An illustration of the benchmark pro-inflammatory protein value set is shown in FIG. 4. Thus, the optimal value range for the level of salivary IL-6 upon waking (0 mins) is given as 7.338 pg/ml to 13.196 pg/ml. Similar optimal value ranges are shown for other preferred time points including 30 minutes post waking (30 mins), 60 minutes post waking (60 mins), 6 hours post waking (6 hrs), 10 hours post waking (10 hrs), and 16 hours post waking (16 hrs). All values are refer to the concentration of IL-6 saliva and are shown in units of pg/mL.

An additional benchmark pro-inflammatory protein value set of values is determined by calculating the slope over time of the levels of IL-6 in the saliva of the 45 individuals described in Example I at time of waking and 30 minutes post waking is established. This data set is also divided into 10^{th} percentiles, and, in one embodiment, the optimal value range is defined as the 40^{th} through 60^{th} percentiles. An illustration of the slope over time benchmark pro-inflammatory protein value set is shown in FIG. 6. Thus, the optimal value range for the slope over time of salivary IL-6 measured at time of waking and 30 minutes post waking is given as −0.277 to −0.124. All values refer to the slope of IL-6 in saliva vs. time and are shown in units pg/(mL*min).

The individual to be assessed, is asked to collect samples of a non-blood body fluid, e.g., saliva, at about the same time points as the levels used to establish the benchmark pro-inflammatory protein value set of values, i.e. at waking, 30 minutes post waking, 60 minutes post waking, 6 hours post waking, 10 hours post waking and 16 hours post waking. He is given a saliva collection kit that contains labeled tubes, instructions, an ice pack and a storage container to take home and use to collect the necessary saliva samples. After completing sample collection, the individual ships his saliva samples to a testing lab for analysis. The lab uses ELISA methodology to measure the level of salivary IL-6, a pro-inflammatory protein, in the individual's saliva samples.

The levels of salivary IL-6 resulting from the ELISA analysis of the individual's saliva samples are then used to calculate and establish two pro-inflammatory protein values: (1) the level of IL-6 in the individual's saliva sample at waking and (2) the slope over time of the levels of IL-6 at time of waking and 30 minutes post waking. Table 3 below summarizes the IL-6 values established for the individual to be assessed.

TABLE 3
Pro-Inflammatory Protein Values of the Individual to Be Assessed

| | |
|---|---|
| Level of Salivary IL-6 Upon Waking | 6.52 pg/Ml |
| Salivary IL-6 Slope from 0 mins to 30 mins | −0.54 pg/mL*min |

The next step is to compare the individual's values to the optimal value ranges shown in FIGS. 4 and 6. The individual to be assessed's level of IL-6 at waking value is 6.52 pg/mL which is outside the optimal value range of 7.338 pg/ml to 13.196 pg/ml. Therefore, the individual's first comparison indicates that he has inferior pro-inflammatory immune health. The individual's salivary IL-6 slope from 0 mins to 30 mins value is −0.54 pg/mL*min which is outside the optimal value range of −0.277 pg/mL*min to −0.124 pg/mL*min. Therefore, the individual's second comparison also indicates that he has inferior pro-inflammatory immune health.

In one embodiment, the method according to the invention further comprises an assessment of the effect on pro-inflammatory immune health of at least one lifestyle factor. In this example, the individual to be assessed completes the St. Mary's Sleep Questionnaire, a psychometric assessment tool, in order to assess the his sleep quantity and quality. The results of the assessment indicates that the individual is not sleeping enough and he is not sleeping deeply which may be contributing to his inferior pro-inflammatory immune health.

In one embodiment, the comparison of the individual's values to the optimal value ranges and the results of his lifestyle factor assessment are formatted into a report. The report is then printed or emailed in order to share it with the individual his doctor or a health consultant.

### Example IV

Recommending a Program to Improve Pro-Inflammatory Immune Health to a Healthy Individual

In one embodiment, the method according to the invention can be used to provide a program to improve or maintain the pro-inflammatory immune health of a healthy individual. If the individual's pro-inflammatory protein value is within the optimal pro-inflammatory protein value range, the recommended program comprises instructions for the individual to continue practicing his or her current lifestyles factors. If the individual's pro-inflammatory protein value is outside the optimal pro-inflammatory protein value range, the recommended program comprises instructions to change current lifestyle factors.

As described in Example IV, the individual to be assessed's pro-inflammatory protein values were outside the optimal pro-inflammatory protein value range. Thus, a program that comprises a recommendation to change current lifestyle factors is recommended to the individual. Since the individual's results from the St. Mary's Sleep Questionnaire, a lifestyle factor assessment, indicate that the individual is not sleeping well, i.e., the quality of sleep is poor and the quantity of sleep is low, it is recommended that the individual try to improve his sleep quality and quantity. Specifically, in this example, it is recommended that the individual start practicing relaxation techniques and breathing techniques as well as to try to increase his amount of sleep.

In one embodiment, recommendations to the individual to be assessed is delivered via a personalized report, such as the example shown in Table 4 below. As shown in Table 4, the report contains the individual's name, gender, age, a summary of lifestyle factors, and a comparison of the individual to be assessed's pro-inflammatory protein values versus the optimal range of pro-inflammatory protein values as well as recommendations for a program to address pro-inflammatory immune health.

TABLE 4
Personalized Immune Health Report
IMMUNE HEALTH PERSONALIZED REPORT

| | | |
|---|---|---|
| Name: The individual | Gender: Male | Age: 46 |
| to be assessed Smith | | |
| Lifestyle Factor: | Poor | Recommendations: |
| Sleep Quality | | Start practicing |
| Lifestyle Factor: | Low | relaxation techniques |
| Sleep Quantity | | Start practicing |
| Pro-inflammatory Protein | Outside the | breathing techniques |
| Value: | optimal value | Try to increase your |
| Level of Salivary IL-6 Upon | range | amount of sleep. |
| Waking | | |
| Pro-inflammatory Protein | Outside the | |
| Value: | optimal value | |
| Slope of Salivary IL-6 Upon | range | |
| Waking to 30 mins. After | | |
| Waking | | |

The personalized report may be provided to the individual to be assessed in a counseling session where a health consultant sits with him in a private or semi-private counseling area, which is a private or semi-private portion of the retail location. The consultant shows the individual to be assessed the recommended program and reviews the program with him. Other options for delivery of the program to the individual include the internet, email, phone or standard or express mail.

What is claimed is:

1. A kit comprising:
a.) at least two saliva sample collection tubes;
b.) instructions on how to collect, store and ship saliva samples, wherein said instructions require the collection of saliva samples at least two time points relative to morning waking.

2. The kit of claim 1, wherein said at least two time points relative to morning waking are selected from the following: upon waking, 30 minutes post waking, 10 hours post waking and 16 hours post waking.

3. The kit of claim 1, wherein said at least two time points relative to morning waking are (1) at or about waking; and (2) about 30 minutes post waking.

4. The kit of claim 1, wherein said at least two time points relative to morning waking are (1) about 10 hours post waking; and (2) about 16 hours post waking.

5. The kit of claim 1 further comprising a questionnaire for assessing lifestyle factors.

6. The method according to claim 5, wherein said lifestyle factor is selected from at least one of the following: (1) the quantity and/or frequency of smoking or chewing any form of tobacco; (2) sleep quantity and quality; (3) alcohol consumption; (4) caffeine consumption; and (5) body weight., wherein said lifestyle factor is selected from at least one of the following: (1) the quantity and/or frequency of smoking or chewing any form of tobacco; (2) sleep quantity and quality; (3) alcohol consumption; (4) caffeine consumption; and (5) body weight.

7. The kit of claim 1 further comprising: a website link, user ID and password for use in accessing results.

8. The kit of claim 5, wherein the website link comprises a questionnaire for assessing lifestyle factors.

9. The method according to claim 6, wherein said lifestyle factor is selected from at least one of the following: (1) the quantity and/or frequency of smoking or chewing any form of tobacco; (2) sleep quantity and quality; (3) alcohol consumption; (4) caffeine consumption; and (5) body weight., wherein said lifestyle factor is selected from at least one of the following: (1) the quantity and/or frequency of smoking or chewing any form of tobacco; (2) sleep quantity and quality; (3) alcohol consumption; (4) caffeine consumption; and (5) body weight.

10. The kit of claim 2 wherein the website is also a point of sale for treatment products, spa services, or combinations thereof.

11. A kit of claim 1 comprising four collection tubes (1) labeled waking,; (2) labeled 30 minutes post waking; (3) labled 10 hours post waking; and (4) labeled 16 hours post waking.

12. A kit of claim 1 wherein the sample collection tubes contain an absorbent material for the collection of saliva.

13. A method for selling a kit according to claim 1, comprising selling said kit through a retail shopping environment.

14. A method for selling a kit according to claim 1, comprising selling said kit through the Internet.

15. A method of selling a kit according to claim 1, comprising offering said kit for sale at a price that is included in the price of one or more of said treatment products, spa services or combination thereof.

16. A method of selling a kit according to claim 1, comprising offering said kit for sale at a price which includes the cost of one or more treatment products or spa services.
